(19) United States
(12) Patent Application Publication
(10) Pub. No.: US 2010/0070022 A1
(43) Pub. Date: Mar. 18, 2010

(54) LAYER BY LAYER MANUFACTURING OF A STENT

(75) Inventor: Michael Kuehling, Munich (DE)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc., Maple Grove, MN (US)

(21) Appl. No.: 12/558,153

(22) Filed: Sep. 11, 2009

Related U.S. Application Data
(60) Provisional application No. 61/096,669, filed on Sep. 12, 2008.

(51) Int. Cl. A61F 2/06; B23P 15/00

(52) U.S. Cl. 623/1.16; 623/1.44; 623/1.42; 623/1.43; 29/460

(57) ABSTRACT

A stent is provided which has a relatively less porous support structure that includes a first set of consolidated particles and at least one relatively more porous reservoir that includes a second set of consolidated particles that differ in composition from the first set of consolidated particles.

### RELATED APPLICATIONS

This application claims priority from U.S. provisional application 61/096,669, filed Sep. 12, 2008, which is incorporated by reference herein in its entirety.

### TECHNICAL FIELD

The invention relates generally to stents, and more particularly to a drug eluting stent that is manufactured in accordance with a layer by layer manufacturing technique.

### BACKGROUND OF THE INVENTION

Stents and stent delivery devices are employed in a number of medical procedures and as such their structure and function are well known. Stents are used in a wide array of bodily vessels including coronary arteries, renal arteries, peripheral arteries including iliac arteries, arteries of the neck and cerebral arteries as well as in other body structures, including but not limited to arteries, veins, biliary ducts, urethras, fallopian tubes, bronchial tubes, the trachea, the esophagus and the prostate.

Stents are typically cylindrical, radially expandable prostheses introduced via a catheter assembly into a lumen of a body vessel in a configuration having a generally reduced diameter, i.e. in a crimped or unexpanded state, and are then expanded to the diameter of the vessel. In their expanded state, stents support or reinforce sections of vessel walls, for example a blood vessel, which have collapsed, are partially occluded, blocked, weakened, or dilated, and maintain them in an open unobstructed state. To be effective, the stent should be relatively flexible along its length so as to facilitate delivery through torturous body lumens, and yet stiff and stable enough when radially expanded to maintain the blood vessel or artery open. Such stents may include a plurality of axial bends or crowns adjoined together by a plurality of struts so as to form a plurality of U-shaped members coupled together to form a serpentine pattern.

Stents may be formed using any of a number of different methods. One such method involves forming segments from rings, welding or otherwise forming the stent to a desired configuration, and compressing the stent to an unexpanded diameter. Another such method involves machining tubular or solid stock material into bands and then deforming the bands to a desired configuration. While such structures can be made many ways, one method is to cut a thin-walled tubular member of a biocompatible material (e.g. stainless steel, titanium, tantalum, super-elastic nickel-titanium alloys, high-strength thermoplastic polymers, etc.) to remove portions of the tubing in a desired pattern, the remaining portions of the metallic tubing forming the stent. Such a method can cut the tubular member using a laser, a chemical etch or an electrical discharge.

### SUMMARY OF THE INVENTION

In accordance with the present invention, a stent is provided. The stent has a relatively less porous support structure that includes a first set of consolidated particles and at least one relatively more porous reservoir that includes a second set of consolidated particles that differ in composition from the first set of consolidated particles.

In accordance with one aspect of the invention, one or more therapeutic agents may be located in pores of the porous reservoir.

In accordance with another aspect of the invention, one or more therapeutic agents may be provided in the pores of the porous reservoir such that the porous reservoir regulates transport of chemical species between the reservoir and an exterior of the stent upon implantation or insertion of the stent into a subject.

In accordance with another aspect of the invention, the support structure may comprise a plurality of struts and the porous reservoir is located in one of the struts.

In accordance with another aspect of the invention, the first set of consolidated particles may be metal or ceramic particles.

In accordance with another aspect of the invention, the second set of consolidated particles may include biodisintegrable particles.

In accordance with another aspect of the invention, the porous reservoirs may be exposed to at least a luminal surface of the strut.

In accordance with another aspect of the invention, at least one porous seal may be located over an exposed surface of the reservoir to further regulate transport of the chemical species between the reservoir and the exterior of the stent.

In accordance with another aspect of the invention, the therapeutic agent may be selected from one or more of the group consisting of anti-thrombotic agents, anti-proliferative agents, anti-inflammatory agents, anti-restenotic agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, TGF-β elevating agents, and agents that interfere with endogenous vasoactive mechanisms.

In accordance with another aspect of the invention, a method of manufacturing a stent is provided. The method includes dividing a three-dimensional pattern of a stent into a series of layers. At least a plurality of the layers includes a relatively less porous region and a relatively more porous region. Each of the layers are sequentially printed, one on top of another, from a plurality of different types of particles. Each of the layers are sequentially compacted and sintered such that the more porous regions of the plurality of layers collectively form a support structure and the less porous regions of the plurality of layers collectively form at least one porous reservoir located in the support structure.

### BRIEF DESCRIPTION OF THE DRAWINGS

FIG. 1 shows a flow chart of a layer manufacturing process that can be used to fabricate objects such as drug eluting stents by the consolidation of particulate or powder layers.

FIG. 2A is a schematic perspective view of a stent in accordance with an embodiment of the invention. FIG. 2B is a schematic cross-sectional view taken along line b-b of FIG. 2A. FIG. 2C is a schematic perspective view of a portion of the stent of FIG. 2A.

FIGS. 3A-3G and 4A-4E are schematic top views illustrating various reservoir configurations and arrays of the same, which may be employed in various embodiments of the invention.

FIGS. 5A-5E are schematic cross-sectional views illustrating various reservoir configurations, which may be employed in various embodiments of the invention.

FIGS. 6a-6i through 10a-10i are schematic cross-sectional views illustrating additional various reservoir configurations, which may be employed in various embodiments of the invention.

FIG. 11 shows one example of a layer manufacturing system that may be used to fabricate stents in accordance with the present invention.

FIG. 12 shows an alternative example of the printing station shown in FIG. 11, which may be employed to print two different types of particles in the same layer.

FIG. 13 is a schematic cross-sectional view of a strut similar to that depicted in FIG. 2B which employ a porous seal over the reservoir.

### DETAILED DESCRIPTION

Manufacturing techniques or technologies generally known as “layered manufacturing” have emerged over the last decade. With the new technique, parts are made by building them up on a layer-by-layer basis. That is, layered manufacturing is an additive fabrication technology. This is essentially the reverse of conventional machining, which is a subtractive fabrication technology since material is removed from a substrate or preform until the final shape is achieved. Layered manufacturing can offer a considerable savings in time, and therefore cost over conventional machining methods such as laser cutting and the like. Moreover, there is a potential for making very complex parts of either solid, hollow or latticed construction, which can be exceedingly difficult with conventional manufacturing techniques. In addition, layer manufacturing can avoid the need for welded joints, which are commonly required in conventional stents and which can be time-consuming to form and may serve as points of failure.

Layer manufacturing methods build an object of any complex shape layer by layer or point by point without using a pre-shaped tool such as a die or mold. The method begins with creating a Computer Aided Design (CAD) file to represent the geometry of a desired object. As a common practice, this CAD file is converted to a stereo lithography (.STL) format in which the exterior and interior surfaces of the object is approximated by a large number of triangular facets that are connected in a vertex-to-vertex manner. A triangular facet is represented by three vertex points each having three coordinate points: (x₁, y₁, z₁), (z₁, y₂, z₂), and (x₃, y₃, z₃). A perpendicular unit vector (i,j,k) is also attached to each triangular facet to represent its normal for helping to differentiate between an exterior and an interior surface. This object geometry file is further sliced into a large number of thin layers with each layer being represented by a set of data points, or the contours of each layer being defined by a plurality of line segments connected to form polylines on an X-Y plane of a X-Y-Z orthogonal coordinate system. The layer data are converted to tool path data normally in terms of computer numerical control (CNC) codes such as O-codes and M-codes. These codes are then utilized to drive a fabrication tool for defining the desired areas of individual layers and stacking up the object layer by layer along the Z-direction. In this way layer manufacturing enables direct translation of the CAD image data into a three-dimensional (3-D) object.

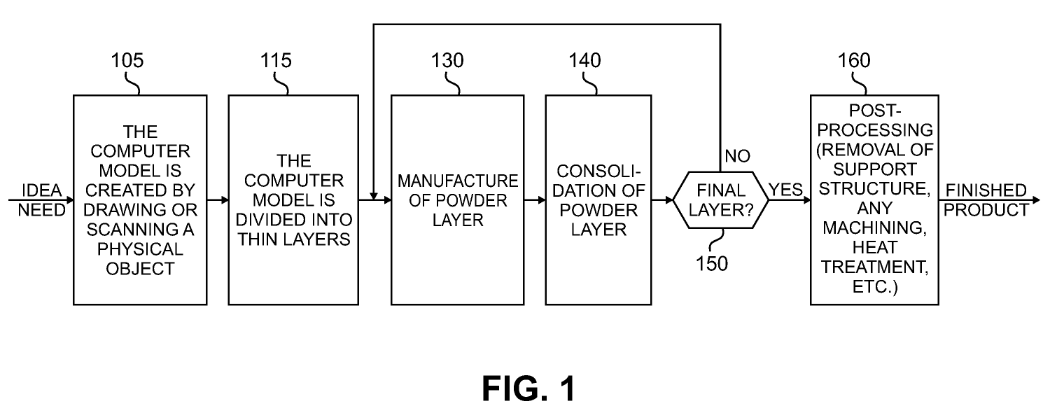

FIG. 1 shows a flow chart of a layer manufacturing process that can be used to fabricate objects such as drug eluting stents by the consolidation of particulate or powder layers. In step 105 a computer model is created by drawing or scanning the physical object. Then, in step 115 the computer model is divided into thin layers, providing a data file containing information on each layer (thickness, shape, materials, etc.) and the relative location of the layers. The fabrication of the object is initiated by sending information on the first layer to a manufacturing unit in step 130. In this unit, a physical particulate layer is constructed (i.e., printed) based on the digital information on the layer. When the particulate layer is complete (it may consist of several materials), it is transported to the compaction unit in step 140 and transformed into a solid material. While the compaction process is proceeding, the manufacturing unit receives information on the next layer and starts to recreate this layer with additional particles. The manufacture and consolidation of the particulate layers are repeated until it is determined at step 150 that the object is finished. Examples of optional post-processing that may be performed in step 160 includes the removal of support particles, heat treatment, or processing using subtractive techniques.

The present invention employs layer manufacturing techniques to the fabrication of a wide variety of stents such as drug eluting stents from consolidated particles, including, without limitation, various balloon-expandable and self-expanding stents, as well as those formed into spiral, coil or woven geometries, either open or closed cell.

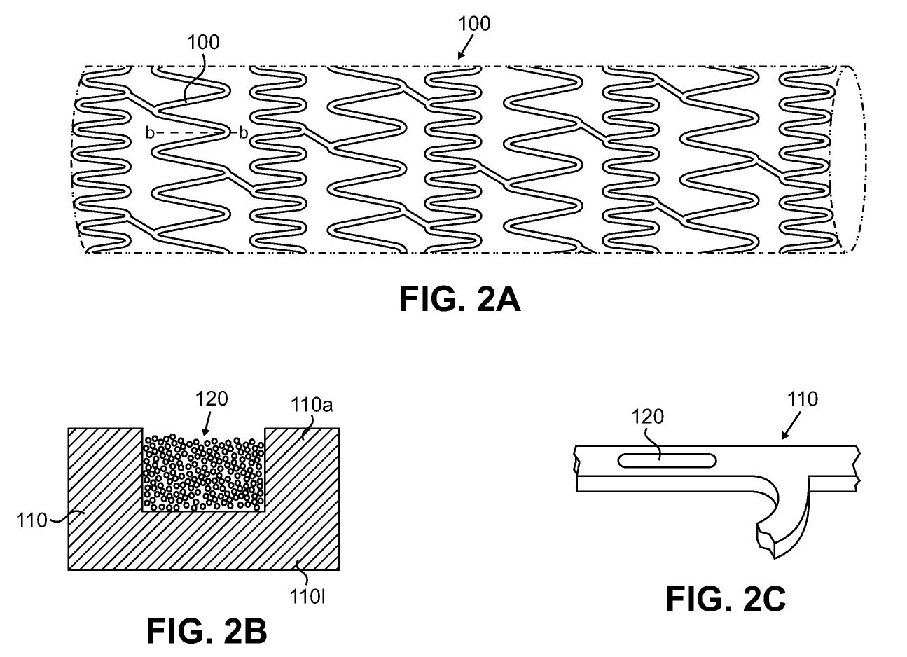

One example of a stent is shown in FIG. 2a. The stent 100 includes a number of interconnected struts 110. The stent may be manufactured layer by layer along the building direction, which is the Z-direction shown in FIG. 2a. That is, each of the layers extends along a plane perpendicular to a longitudinal axis of the stent. Alternatively, the stent 100 may be manufactured as a flat sheet that is formed in a layer by layer manner. The flat sheet is subsequently rolled into a tubular configuration to form the stent.

FIG. 2B is a cross-section taken along line b-b of strut 110 of stent 100 of FIG. 2A, which has an abluminal surface 100a and a luminal surface 100l. The strut 110 includes a porous reservoir 120 that can be filled with a therapeutic-agent-containing composition. In this example the porous reservoir 120 is provided within the abluminal surface of the strut 110. Alternatively, the porous reservoir 120 may be provided within the luminal surface of the strut 110. As another alternative, among others, porous reservoirs 120 may be provided within each of the luminal and abluminal surfaces of the tubular strut 110. One or more such porous reservoirs 120 may be provided in each of the struts 110 in the stent 100 or in selected ones of the struts 110. FIG. 2C is a perspective view of a portion of strut 110s to shown the shape of the porous reservoir 120.

The therapeutic-agent-containing composition that is loaded into the porous reservoirs 120 may consist essentially of one or more therapeutic agents, or it may contain further optional agents such as polymer matrix materials, diluents, excipients or fillers. Moreover, all of the porous reservoirs 120 may be filled with the same therapeutic-agent-containing composition, or some porous reservoirs may be filled with a first therapeutic-agent-containing composition while other porous reservoirs may be filled with a different therapeutic-agent-containing composition, among other possibilities. For example, it is possible to provide one or more first porous reservoirs that are filled with a first therapeutic agent (e.g., an anti-inflammatory agent, an endothelialization promoter or an antithrombotic agent) at the inner, luminal surface of the strut 110, and one or more second porous reservoirs filled with a second therapeutic agent that differs from the first therapeutic agent (e.g., an anti-restenotic agent) at the outer, abluminal surface of the strut 110.

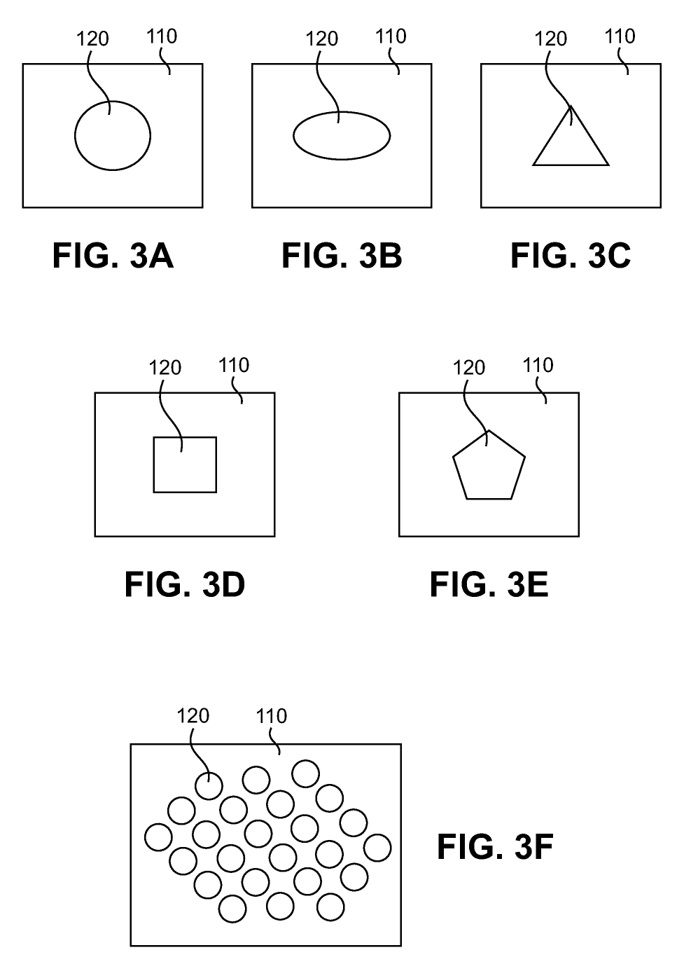

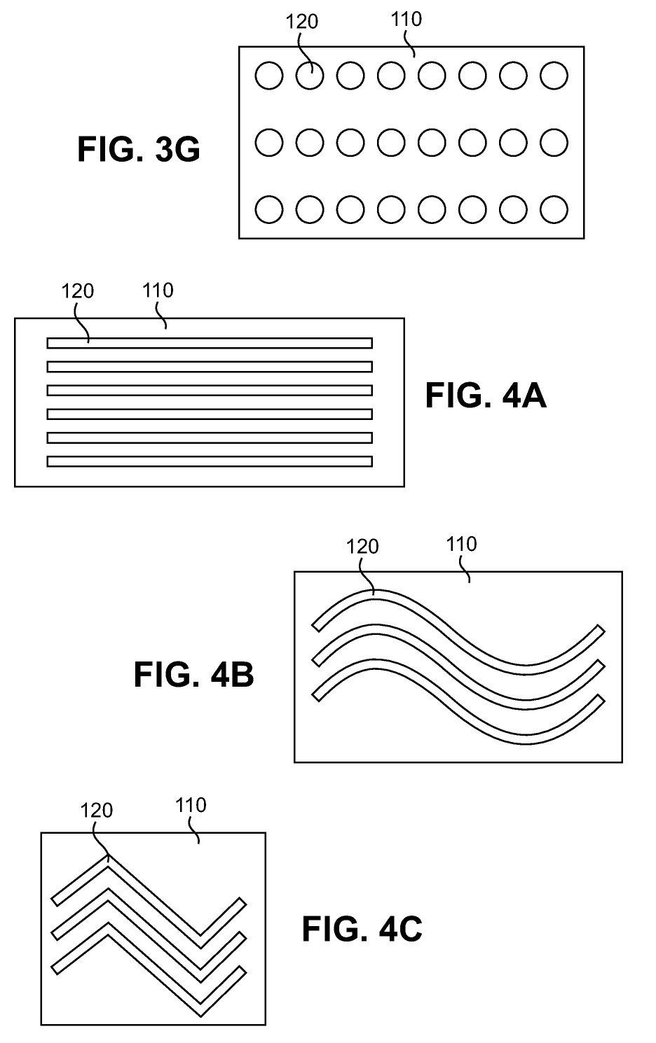

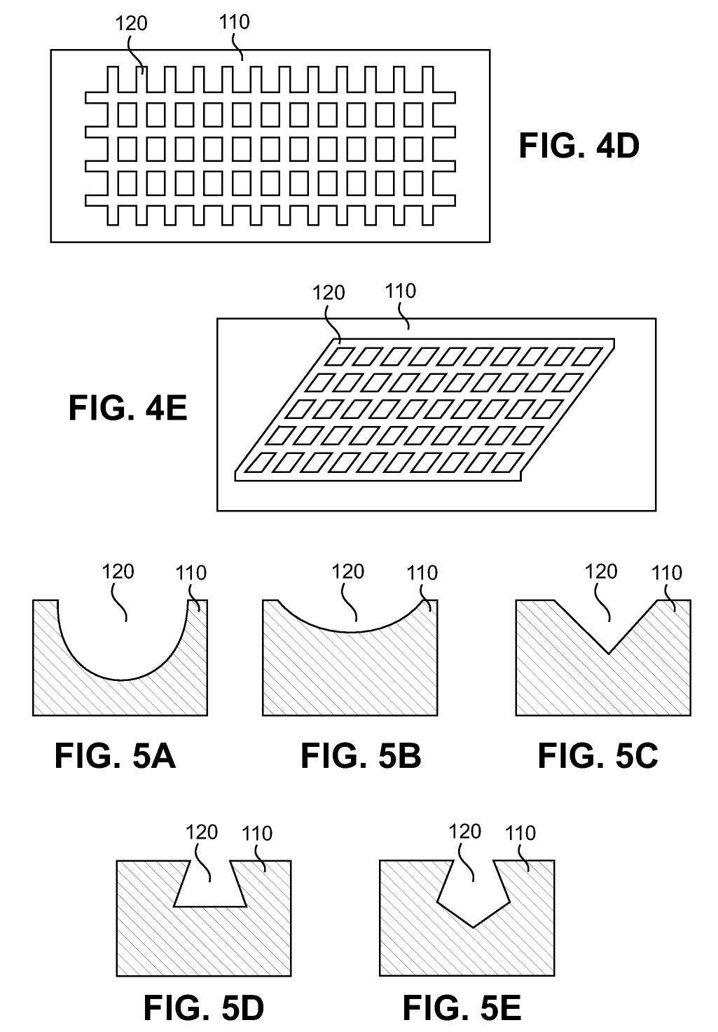

The porous reservoirs 120 which contain the therapeutic agents may come in various shapes and sizes. Examples include regions whose lateral dimensions are circular (see, e.g., the top view of the circular hole of FIG. 3A, in which the porous reservoirs 110d within the stent 110 is designated with a darker shade of grey), oval (see FIG. 3B), polygonal, for instance triangular (see FIG. 3C), quadrilateral (see FIG. 3D), penta-lateral (see FIG. 3E), as well as porous reservoirs of various other regular and irregular shapes and sizes. Multiple porous reservoirs can be provided in a near infinite variety of arrays. See, e.g., the porous reservoirs shown in FIGS. 3F and 3G. Further examples of porous reservoirs 120 include trenches, such as simple linear trenches (see FIG. 4A), wavy trenches (see FIG. 4B), trenches formed from linear segments whose direction undergoes an angular change (see FIG. 4C), trench networks intersecting at right angles (see FIG. 4D), as well as other angles (see FIG. 4E), as well as other regular and irregular trench configurations.

The therapeutic agent-containing porous reservoirs can be of any size. Commonly, stents contain therapeutic agent-containing porous reservoirs whose smallest lateral dimension (e.g., the diameter for a cylindrical region, the width for an elongated region such a trench, etc.) is less than 10 mm (10000 μm), for example, ranging from 10,000 μm to 5000 μm to 2500 μm to 1000 μm to 500 μm to 250 μm to 100 μm to 50 μm to 10 μm to 5 μm to 2.5 μm to 1 μm or less.

As indicated above, the porous reservoirs 120 may be in the form of blind holes, through-holes, trenches, etc. Such reservoirs 120 may have a variety of cross-sections, such as semicircular cross-sections (see, e.g., FIG. 5A), semi-oval cross-sections (see, e.g., FIG. 5B), polygonal cross-sections, including triangular (see, e.g., FIG. 5C), quadrilateral (see, e.g., FIG. 5D) and penta-lateral (see, e.g., FIG. 5E) cross-sections, as well as other regular and irregular cross-sections. In certain embodiments, the porous reservoirs are high aspect ratio porous reservoirs, meaning that the depth of the reservoir is greater than the width of the reservoir, for example, ranging from 1.5 to 2 to 2.5 to 5 to 10 to 25 or more times the width. In certain other embodiments, the porous reservoirs are low aspect ratio porous reservoirs, meaning that the depth of the reservoir is less than the width of the reservoir, for example, ranging from 0.75 to 0.5 to 0.4 to 0.2 to 0.1 to 0.04 or less times the width.

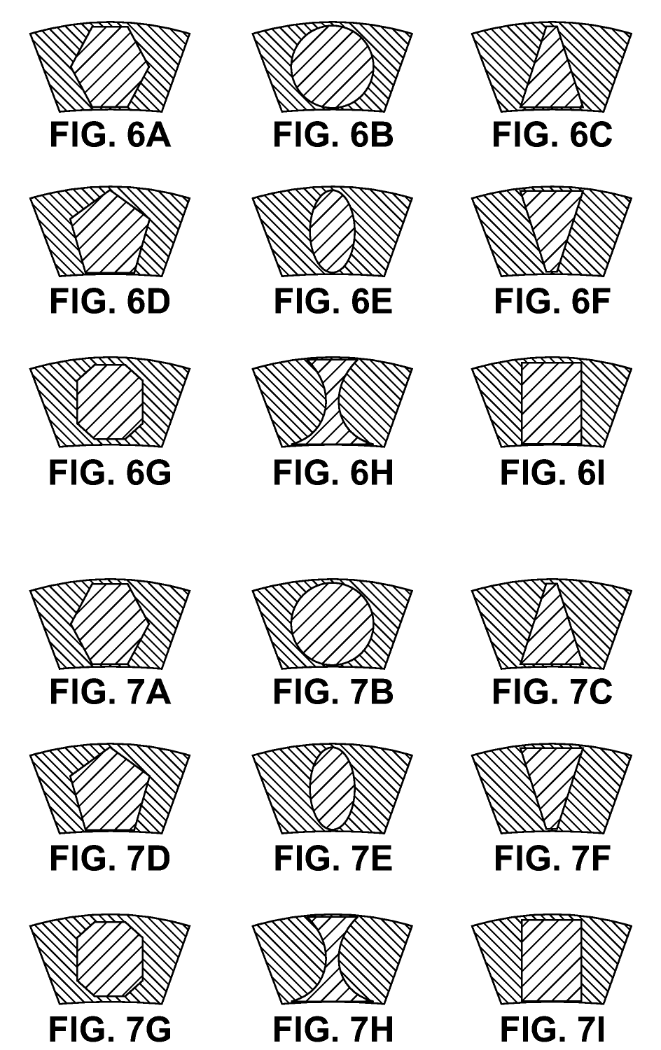

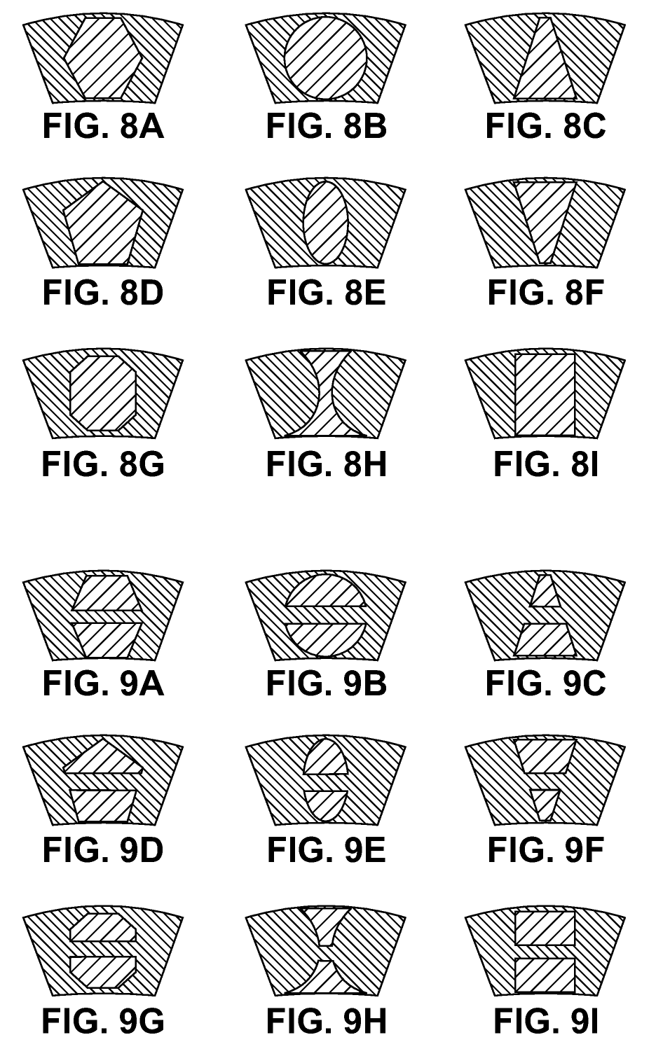

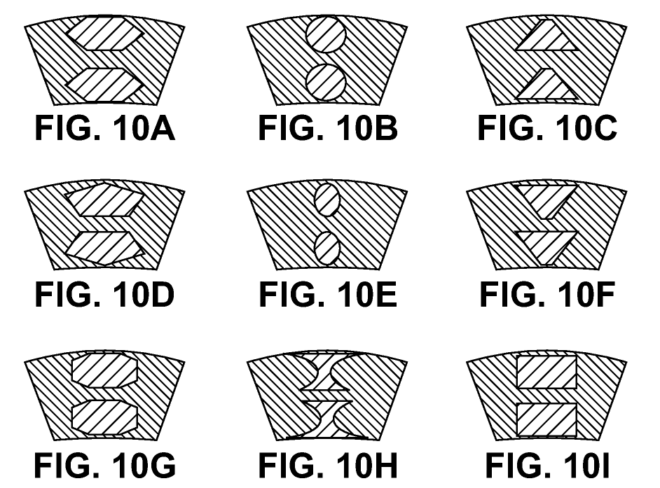

The cross-sections of additional illustrative porous reservoirs are shown in FIGS. 6-10. In FIGS. 6-8 a single porous reservoir 120 is provided in each cross-section, which is exposed to both the luminal and abluminal surfaces of the strut. In FIGS. 9 and 10 two porous reservoirs 120 are provided in each cross-section, one exposed to the luminal surface of the strut and the other exposed to the abluminal surface of the strut.

By varying the size (i.e., volume) and number of the porous reservoirs, as well as the concentration of the therapeutic agents within the porous reservoirs, a range of therapeutic agent loading levels can be achieved. The amount of loading may be determined by those of ordinary skill in the art and may ultimately depend, for example, upon the disease or condition being treated, the age, sex and health of the subject, the nature (e.g., potency) of the therapeutic agent, or other factors.

A wide variety of particulate or powder materials may be used to form a stent that is fabricated in accordance with layer manufacturing techniques. Examples include one or more of the following: biostable and biodisintegrable substantially pure metals, including gold, niobium, platinum, palladium, iridium, osmium, rhodium, titanium, zirconium, tantalum, tungsten, niobium, ruthenium, magnesium, zinc and iron, among others, and biostable and biodisintegrable metal alloys, including metal alloys comprising iron and chromium (e.g., stainless steels, including platinum-enriched radiopaque stainless steel), niobium alloys, titanium alloys, nickel alloys including alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N), alloys comprising cobalt, chromium, tungsten and nickel (e.g., L605), and alloys comprising nickel and chromium (e.g., inconel alloys), and biodisintegrable alloys including alloys of magnesium, zinc and/or iron (and their alloys with combinations of each other an Ce, Ca, Zr and Li), among others. Further examples, not necessarily exclusive of the foregoing, include the biodegradable metallic materials described in U.S. Patent App. Pub. No. 2002/0004060 A1, entitled “Metallic implant which is degradable in vivo.” These include substantially pure metals and metal alloys whose main constituent is selected from alkali metals, alkaline earth metals, iron, and zinc, for example, metals and metal alloys containing magnesium, iron or zinc as a main constituent and one or more additional constituents selected from the following: alkali metals such as Li, alkaline-earth metals such as Ca and Mg, transition metals such as Mn, Co, Ni, Cr, Cu, Cd, Zr, Ag, Au, Pd, Pt, Re, Fe and Zn, Group IIIa metals such as Al, and Group IVa elements such as C, Si, Sn and Pb.

In some cases the stent may be formed from two or more different materials. For example, one section of the stent may comprise a flexible material such as stainless steel or a shape memory alloy such as Nitinol, while another section may be formed of a more rigid, radiopaque material such as gold, tantalum, platinum, and so forth, or alloys thereof.

Another class of particulate material that may be used to form the stent include ceramic materials, including, for example, silicon-based ceramics, such as those containing silicon nitrides, silicon carbides and silicon oxides (sometimes referred to as glass ceramics), calcium phosphate ceramics (e.g., hydroxyapatite) and carbon and carbon-based, ceramic-like materials such as carbon nitrides.

In layer manufacturing, two main groups of particles or powder are used for the manufacture of the particulate layers; building particles or powder and support particles or powder. The building particles of each layer forms a thin slice of the product being constructed (transformed to a solid material). In order to be able to build stents having a wide variety of shapes (overhang, inner geometries, etc.), it is often necessary to support the stent during the building process. To this end, support particles can be used that do not sinter in the consolidation process, but serves to provide support during the building process. The support particles are compacted, whereas the building particles are compacted and sintered to form the consolidated particles in the particulate layers. In the construction of metallic objects, the support particles are typically a ceramic material, or a mixture of ceramic materials. The sintering temperature of the support particles must be substantially higher than the sintering temperature of the building particles, so that the support particles are not sintered but may be easily removed when the entire stent is finished. The particles in the support particles typically have an irregular shape in order for the support particles to obtain a high strength on compaction.

A particle layer may contain several kinds of building particles, both with respect to material and particle structure. This allows for the production of stents with custom properties for given applications. For example, one portion of a layer may supply stiffness and rigidity while another portion supplies sufficient flexibility to facilitate delivery of the stent through body lumens, while yet another portion has a porosity sufficient to contain the therapeutic agent-containing composition. A gradual transition between materials (graded materials) can be obtained by increasing the portion of a new material for each new layer being compacted. In this manner, problems associated with differing properties of the two materials are avoided, e.g. the coefficient of thermal expansion. As another example, portions of a layer that will be most subject to wear resistance may be hardened by adding ceramic particles to the building particles. This part of the stent may then comprise ceramic particles bound together by metallic material.

The particles used in the building particles may have different characteristics (size, shape, structure). This makes it possible to control the after-consolidation porosity of the layer so as to form, for example, the porous reservoirs 120 shown in FIG. 1b. The density of a layer is determined by the consolidation parameters, particle material, and particle characteristics.

The consolidated particles in the porous reservoirs can serve to regulate transport of chemical species (e.g., in many embodiments, the therapeutic agent, among others) between the porous reservoirs and the exterior of the stent. The consolidated particles in the porous reservoirs may be biodisintegrable particles (i.e., materials that, upon placement in the body, are dissolved, degraded, eroded, resorbed, and/or otherwise removed from the placement site over the anticipated placement period) such as biodisintegrable metallic particles. As the particles disintegrate, the rate of transport of the chemical species between the porous reservoirs and the exterior of the stent increases in a manner that can be controlled by choosing the type, size, packing and layer thickness of the particle layer. If the particles in the porous reservoirs are disintegrable, the release rate or rate profile of the therapeutic agent is determined both by the porosity and the rate of disintegration of the consolidated particles in the porous reservoirs. The size of the pores in the porous reservoirs may be chosen to achieve a desired release rate of the therapeutic agent. In some embodiments pore sizes may range, for example, from nanopores (i.e., pores having widths of 50 nm or less), which include micropores (i.e., pores having widths smaller than 2 nm) and mesopores (i.e., pores having widths ranging from 2 to 50 nm), to macropores (i.e., pores having widths that are larger than 50 nm).

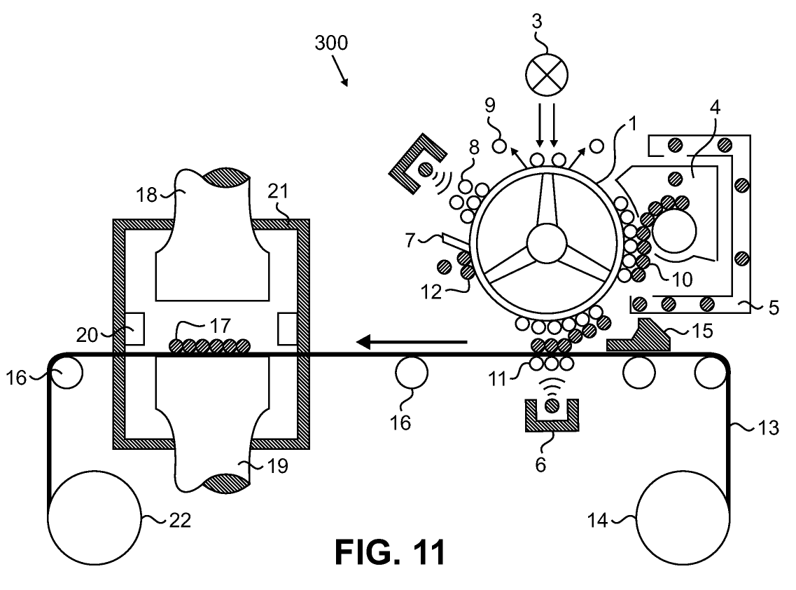

FIG. 11 shows one example of a layer manufacturing system 300 that may be used to fabricate stents in accordance with the present invention. The system 300 includes a printing station, a particle compaction station, and a transport device for conveying individually printed layers from the printing station to the compaction station.

The printing station includes a cylindrical particle receptor 1, which rotates clockwise with a constant rotational speed. A primary corona wire (not shown) charges the particle receptor 1, as indicated by the ionized gas molecules 8 attached to the surface of the particle receptor 1. A light emitting rod 3 illuminates the particle receptor in accordance with the pattern of the next stent layer to be fabricated. Light emitting rod 3 is typically an LED type printer head that includes many small light emitting diodes that are arranged to illuminate the particle receptor 1 with the relevant pattern. The light emitting rod 3 and the particle receptor 1 are closely spaced from one another so that a difference in surface potential can be achieved between the illuminated and non-illuminated areas of the particle receptor 1. In FIG. 11 this is indicated by showing that certain gas molecules 9 become detached from the particle receptor. When the illuminated particle receptor 1 passes the feed entry from a particle magazine 4, particles 10 will be electrostatically attracted to the receptor 1 in accordance with the illumination pattern. Particles that do not attach to receptor 1 falls into a tray 5 and is returned to particle magazine 4.

While the printing station described above electrostatically attracts the particles to a photoreceptor, in other cases the particles may be attracted to a receptor by other means, such as with an adhesive, for example.

The transport device comprises a conveyor belt 13 that rolls off a supply reel 14 and onto a collector reel 22. The movement of the conveyor belt 13 is synchronized with the rotation of particle receptor 1 so that the mutual relation between the individual particles is maintained during deposition from particle receptor 1 to conveyor belt 13, ensuring that the pattern formed by the particles on the cylindrical particle receptor 1 is maintained on the conveyor belt's planar surface. Immediately upstream from the particle receptor 1 a preheating element 15 may be arranged in close proximity to the conveyor belt 13, which serves to enhance the adhesiveness of the conveyor belt 13. A secondary corona wire 6 is located below the convey belt 13, directly beneath the power receptor 1. The secondary corona wire 6 generates ionized gas molecules 11 at the lower surface of conveyor belt 13. The adhesion forces between gas molecules 11 and particles 10 are larger than the forces holding the particles to particle receptor 1. In this way the particle pattern of particle receptor 1 is transferred to conveyor belt 13. Particles 12 that might remain on the receptor after having passed over conveyor belt 13 are removed with a scraper device 7 or the like. When the particles constituting a complete layer has been deposited on the conveyor belt 13 as described above, it is transported to the sintering die for deposition and consolidation, i. e. sintering. The conveyor belt 13 should be sufficiently rigid so that the particles deposited thereon will not be displaced during transportation. To this end it may include perforations along its sides to ensure even movement thereof. The conveyor belt 13 should be formed from a material that decomposes at or below the relevant sintering temperatures, and it should decompose without leaving behind any harmful residual material in the fully formed stent.

The compacting station includes a housing 21 in which pistons 18 and 19 are employed to exert pressure on the power layers. The compacting station also includes an energy source or sources 17 for subjecting the particle layers to the elevated temperatures necessary to perform sintering. The energy source 17 may be thermal, electrical, microwave or the like. The appropriate energy source that is used will often depend on the natures of the particle materials that are employed. For instance, an electrical source will often be suitable for metallic particles, which are electrically conductive, whereas a microwave source may be suitable for particles that are not electrically conductive, such as ceramic materials. The sintering temperature that needs to be achieved will depend on the particular building material being used, but will often be in the range of about 60 to 80% of the melting temperature of the building material, as measured on the Celsius scale. The lower piston 19 will gradually or stepwise be lowered as new particle layers are deposited and the height of the stent under manufacture correspondingly increases. In this way the path of the conveyor belt 13 may remain unchanged regardless of the height of the gradually growing stent.

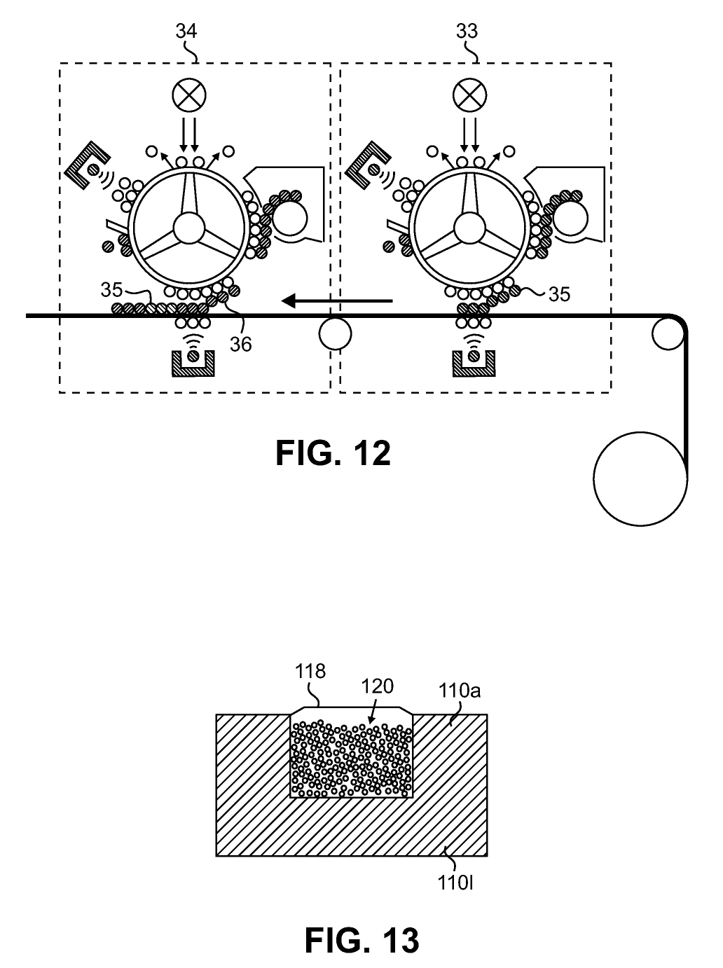

FIG. 12 shows how two different particle materials may be deposited (sequentially) in the same layer. One particle composition 35 (e. g. a metallic one) is deposited from a first particle receptor in a first printing station 33 and the layer is supplemented with a second layer 36 (e.g. a ceramic one) from a different particle receptor in a second printing station 34. In this way different parts of any given layer may be supplied with different types of building particles.

As noted in FIG. 1, various post-processing may be performed after compacting the layers to form the stent. For example, as shown in FIG. 13, in some cases a porous seal 118 may be provided over the porous reservoirs to add further stability and delay the rate at which the therapeutic agent-containing composition is released. If the consolidated particles in the porous reservoirs are biodisintegrable, the seal may also help control the rate at which the particles disintegrates, thereby further regulating rate at which the therapeutic agent-containing composition is released. In some embodiments the seal may comprise nanopores (commonly at least 10⁶, 10⁹, 10¹² or more nanopores per cm²), a microporous surface, which is one that comprises micropores, a mesoporous surface, which is one that comprises mesopores, or a macroporous surface, which is one that comprises macropores. The seal 118 can be laser welded, fused or otherwise secured over the porous reservoir by any appropriate means.

“Biologically active agents,” “drugs,” “therapeutic agents,” “pharmaceutically active agents,” “pharmaceutically active materials,” and other related terms may be used interchangeably herein and include genetic therapeutic agents, non-genetic therapeutic agents and cells. A wide variety of therapeutic agents can be employed in conjunction with the present invention. Numerous therapeutic agents are described below.

Suitable non-genetic therapeutic agents for use in the porous reservoirs may be selected, for example, from one or more of the following: (a) anti-thrombotic agents such as heparin, heparin derivatives, urokinase, clopidogrel, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promoters; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) smooth muscle relaxants such as alpha receptor antagonists (e.g., doxazosin, tamsulosin, terazosin, prazosin and alfuzosin), calcium channel blockers (e.g., verapimil, diltiazem, nifedipine, nicardipine, nimodipine and bepridil), beta receptor agonists (e.g., dobutamine and salmeterol), beta receptor antagonists (e.g., atenolol, metaprolol and butoxamine), angiotensin-II receptor antagonists (e.g., losartan, valsartan, irbesartan, candesartan, eprosartan and telmisartan), and antispasmodic/anticholinergic drugs (e.g., oxybutynin chloride, flavoxate, tolterodine, hyoscyamine sulfate, diclomine), (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, AII, AIII, AIV, AV, etc.), (z) selective estrogen receptor modulators (SERMs) such as raloxifene, lasofoxifene, arzoxifene, miproxifene, ospemifene, PKS 3741, MF 101 and SR 16234, (aa) PPAR agonists such as rosiglitazone, pioglitazone, netoglitazone, fenofibrate, bexaotene, metaglidasen, rivoglitazone and tesaglitazar, (bb) prostaglandin E agonists such as alprostadil or ONO 8815Ly, (cc) thrombin receptor activating peptide (TRAP), (dd) vasopeptidase inhibitors including benazepril, fosinopril, lisinopril, quinapril, ramipril, imidapril, delapril, moexipril and spirapril, (ee) thymosin beta 4, and (ff) phospholipids including phosphorylcholine, phosphatidylinositol and phosphatidylcholine.

Preferred non-genetic therapeutic agents include taxanes such as paclitaxel (including particulate forms thereof, for instance, protein-bound paclitaxel particles such as albumin-bound paclitaxel nanoparticles, e.g., ABRAXANE), sirolimus, everolimus, tacrolimus, zotarolimus, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well derivatives of the forgoing, among others.

Suitable genetic therapeutic agents for use in connection with the present invention include anti-sense DNA and RNA as well as DNA coding for the various proteins (as well as the proteins themselves) and may be selected, for example, from one or more of the following:: (a) anti-sense RNA, (b) tRNA or rRNA to replace defective or deficient endogenous molecules, (c) angiogenic and other factors including growth factors such as acidic and basic fibroblast growth factors, vascular endothelial growth factor, endothelial mitogenic growth factors, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers such as polyvinylpyrrolidone (PVP), SP1017 (SUPRATEK), lipids such as cationic lipids, liposomes, lipoplexes, nanoparticles, or microparticles, with and without targeting sequences such as the protein transduction domain (PTD).

Cells for use in conjunction with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

Further therapeutic agents, not necessarily exclusive of those listed above, have been identified as candidates for vascular treatment regimens, for example, as agents targeting restenosis (anti-restenotic agents). Suitable agents may be selected, for example, from one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists such as bosentan, sitaxsentan sodium, atrasentan, endonentan, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK (D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, atorvastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD (orgotein), SOD mimics, verteporfin, rostaporfin, AGI 1067, and M 40419, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) matrix metalloprotease (MMP) pathway inhibitors such as marimastat, ilomastat, metastat, batimastat, pentosan polysulfate, rebimastat, incyclinide, apratastat, PG 116800, RO 1130830 or ABT 518, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate , nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), rapamycin (sirolimus) and its analogs (e.g., everolimus, tacrolimus, zotarolimus, etc.), cerivastatin, flavopiridol and suramin, (aa) matrix deposition/organization pathway inhibitors such as halofuginone or other quinazolinone derivatives, pirfenidone and tranilast, (bb) endothelialization facilitators such as VEGF and RGD peptide, (cc) blood rheology modulators such as pentoxifylline and (dd) glucose cross-link breakers such as alagebrium chloride (ALT-711).

Numerous additional therapeutic agents for the practice of the present invention may be selected from suitable therapeutic agents disclosed in U.S. Pat. No. 5,733,925 to Kunz.

Although various embodiments are specifically illustrated and described herein, it will be appreciated that modifications and variations of the present invention are covered by the above teachings and are within the purview of the appended claims without departing from the spirit and intended scope of the invention.

1. A stent, comprising:
a relatively less porous support structure that includes a first set of consolidated particles and at least one relatively more porous reservoir that includes a second set of consolidated particles that differ in composition from the first set of consolidated particles.

2. The stent of claim 1 further comprising one or more therapeutic agents located in pores of the porous reservoir.

3. The stent of claim 2 wherein the one or more therapeutic agents are provided in the pores of the porous reservoir such that the porous reservoir regulates transport of chemical species between the reservoir and an exterior of the stent upon implantation or insertion of the stent into a subject.

4. The stent of claim 1 wherein the support structure comprises a plurality of struts and the porous reservoir is located in one of the struts.

5. The stent of claim 1 wherein the first set of consolidated particles are metal or ceramic particles.

6. The stent of claim 1 wherein the second set of consolidated particles includes biodisintegrable particles.

7. The stent of claim 1 wherein the porous reservoirs are exposed to at least a luminal surface of the strut.

8. The stent of claim 1 further comprising at least one porous seal located over an exposed surface of the reservoir to further regulate transport of the chemical species between the reservoir and the exterior of the stent.

9. The stent of claim 2 wherein said therapeutic agent is selected from one or more of the group consisting of anti-thrombotic agents, anti-proliferative agents, anti-inflammatory agents, anti-restenotic agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, TGF-β elevating agents, and agents that interfere with endogenous vasoactive mechanisms.

10. A method of manufacturing a stent, comprising:
dividing a three-dimensional pattern of a stent into a series of layers, at least a plurality of the layers including a relatively less porous region and a relatively more porous region;
sequentially printing each of the layers, one on top of another, from a plurality of different types of particles; and
sequentially compacting and sintering each of the layers such that the more porous regions of the plurality of layers collectively form a support structure and the less porous regions of the plurality of layers collectively form at least one porous reservoir located in the support structure.

11. The method of claim 10 wherein the plurality of different particles include a first particulate composition used to print the relatively more porous regions and a second particulate composition used to print the relatively less porous regions, wherein the first particulate composition is different from the second particulate composition.

12. The method of claim 11 wherein at least one of the first and second particulate compositions comprises metallic particles.

13. The method of claim 11 wherein the second particulate composition comprises biodisintegrable particles.

14. The method of claim 10 wherein the support structure comprises a series of interconnected struts, said porous reservoir being located in one of the struts.

15. The method of claim 10 wherein the porous reservoir is exposed to at least a luminal or abluminal surface of the strut.

16. The method of claim 10 further comprising introducing one or more therapeutic agents into the reservoir.

17. The method of claim 16 further comprising applying a porous seal over the reservoir to regulate transport of the one or more therapeutic agents between the reservoir and an exterior of the stent.

18. The method of claim 10 further comprising rolling the compacted and sintered layers into a tubular shape.

19. The method of claim 10 wherein each of the layers extends along a plane perpendicular to a longitudinal axis of the stent.

20. The method of claim 10 wherein sequentially printing each of the layers includes electrostatically attracting the particles to a layer pattern formed on a photoreceptor.
